(19) United States
(12) Patent Application Publication
(10) Pub. No.: US 2020/0048164 A1
(43) Pub. Date: Feb. 13, 2020

(54) STABLE CATALYSTS FOR OXIDATIVE COUPLING OF METHANE

(71) Applicant: SABIC Global Technologies B.V., Bergen op Zoom (NL)

(72) Inventors: Sagar Sarsani, Sugar Land, TX (US); Wugeng Liang, Sugar Land, TX (US); Dick Nagaki, Sugar Land, TX (US); Krishnan Sankaranarayanan, Sugar Land, TX (US); David West, Sugar Land, TX (US); Aghaddin Mamedov, Sugar Land, TX (US)

(21) Appl. No.: 16/343,095

(22) PCT Filed: Oct. 17, 2017
(86) PCT No.: PCT/IB2017/056449

§ 371 Date: Apr. 18, 2019

Related U.S. Application Data
(60) Provisional application No. 62/411,158, filed on Oct. 21, 2016.

(51) Int. Cl. C07C 2/84; C07C 9/06

(52) U.S. Cl. CPC C07C 2/84; C07C 2523/10; C07C 2523/02; C07C 9/06

(57) ABSTRACT

A method of selecting a stable mixed metal oxide catalyst for an oxidative coupling of methane (OCM) reaction is disclosed. The method may include, obtaining a mixed metal oxide material having catalytically active metal oxides for the OCM reaction and identifying the Tammann temperature (TTam) of at least one of the catalytically active metals oxides of the mixed metal oxide material. The method further includes selecting the mixed metal oxide material for use as a catalyst in the OCM reaction if the at least one catalytically active metal oxides present in the mixed metal oxide material has a TTam greater than a predetermined temperature.

### CROSS REFERENCE TO RELATED APPLICATIONS

This application claims the benefit of priority of U.S. Provisional Patent Application No. 62/411,158 filed Oct. 21, 2016, which is hereby incorporated by reference in its entirety.

### FIELD OF INVENTION

The present invention relates to oxidative coupling of methane to form hydrocarbons with two or more carbon atoms. More specifically, the present invention relates to catalysts that are stable at optimum temperatures for carrying out the oxidative coupling of methane reaction.

### BACKGROUND OF THE INVENTION

Methane (CH₄) is the simplest alkane, having a single carbon atom bonded to four hydrogen atoms. Methane occurs naturally and abundantly in the earth in the form of natural gas and is often used as fuel. In addition to its use as fuel, methane is converted to hydrocarbons having two or more carbon atoms (C₂₊), which are more conducive as building blocks for other petrochemical products. Oxidative coupling of methane (OCM) is the chemical reaction by which methane is converted to C₂₊ hydrocarbons. One of the more common products of oxidative coupling of methane is ethylene, the reaction for which is illustrated below:

2CH₄+O₂→C₂H₄+2H₂O

The use of catalysts in oxidative coupling of methane has been studied extensively for decades. Yet, it has been difficult to identify catalysts that provide the appropriate selectivity in reactions so as to make the oxidative coupling of methane sufficiently economical. Many catalyst compositions for the oxidative coupling of methane, including many mixed metal oxide catalysts, have been studied. One of the most widely studied mixed metal oxide catalysts is a sodium (Na), tungsten (W)-manganese (Mn) oxide on a silica (SiO₂) support (Na₂WO₄—Mn—O/SiO₂). In a publication of one such study, “Oxidative Coupling of Methane over Oxide-Supported Sodium-Manganese Catalysts,” Wang et. al., Journal of Catalysis, 155, 390-402 (1995), it was disclosed that at a feed ratio of CH₄/O₂ of 7.44, on Na₂WO₄—Mn—O/SiO₂ catalyst, the best conversion of methane achieved is approximately 20% at approximately 80% C₂ selectivity. Oxidative coupling of methane reaction on a catalyst is often carried out at temperatures of 800° C. to 900° C. But it is known that at these high operating temperatures, the Na₂WO₄—Mn—O/SiO₂ catalyst loses activity with time due to loss of Na and W from the catalyst. The longest reported lifetime of this catalyst in the oxidative coupling of methane reaction is approximately 500 hours. Despite the extensive studies of catalysts for use in the oxidative coupling of methane reaction, there still exists a need for a catalyst that is stable enough to have a life in excess of 500 hours.

### BRIEF SUMMARY OF THE INVENTION

A discovery has been made that provides a solution to the aforementioned issues of low catalyst life for catalysts used in the oxidative coupling of methane reaction. The discovery is premised on judiciously selecting mixed metal oxide materials for the oxidative coupling of methane reaction so that the catalyst is stable for a long period (e.g., greater than 500 hours, preferably greater than 1000 hours, or more preferably greater than 1500 hours). Embodiments of the discovered process may involve selecting mixed metal oxide materials having catalytically active metal oxides with Tammann temperature (T_{Tam}) above a pre-determined amount for use in oxidative coupling of methane catalysts. In this way, stable performance of the mixed metal oxide catalyst in the oxidative coupling of methane reaction can be achieved.

Embodiments of the invention include a method of selecting a stable mixed metal oxide catalyst for an oxidative coupling of methane reaction. The method may include obtaining a mixed metal oxide material having one or more catalytically active metal oxides for the oxidative coupling of methane reaction and identifying the Tammann temperature of one or more of the catalytically active metal oxides. The method further includes selecting the mixed metal oxide material for use as a catalyst in the oxidative coupling of methane reaction if the one or more catalytically active metal oxides present in the mixed metal oxide material has a Tammann temperature greater than 750° C.

Embodiments of the invention include a method of making a stable mixed metal oxide catalyst for an oxidative coupling of methane reaction. The method may include selecting at least a first metal oxide material and a second metal oxide material based on the Tammann temperature of the corresponding metal oxides. The Tammann temperature of one or more corresponding metal oxides may be greater than 750° C. The method may further include combining the first metal oxide material and the second metal oxide material to form a stable material and calcining the stable material to obtain a mixed metal oxide catalyst that can be used in the oxidative coupling of methane reaction.

The following includes definitions of various terms and phrases used throughout this specification.

The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

The terms “wt. %”, “vol. %”, or “mol. %” refers to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The methods of the present invention can “comprise,” “consist essentially of,” or “consist of” particular ingredients, components, compositions, etc., disclosed throughout the specification. With respect to the transitional phrase “consisting essentially of,” in one non-limiting aspect, a basic and novel characteristic of a method of the present invention is the ability to select a stable mixed metal oxide catalyst for an oxidative coupling of methane based on identification of a Tammann temperature (T_{Tam}) of at least one of the catalytically active metals oxides of the mixed metal oxide material.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

For a more complete understanding of the present invention, reference is now made to the following descriptions taken in conjunction with the accompanying drawings, in which:

FIG. 1 shows a method of selecting a stable mixed metal oxide catalyst for an oxidative coupling of methane reaction, according to embodiments of the invention;

FIG. 2 shows a method of making a stable mixed metal oxide catalyst for an oxidative coupling of methane reaction, according to embodiments of the invention; and

FIG. 3 illustrates the performance of SrCeYb oxide catalyst with time on a feed stream of CH₄/O₂.

### DETAILED DESCRIPTION OF THE DISCLOSURE

The invention provides for an efficient selection process to identify and prepare oxidative coupling of methane catalysts that have a substantially longer lifetime when compared with currently known OCM catalysts. As illustrated in non-limiting embodiments in the Examples section, this selection process allows for the identification and preparation of such catalysts that remain catalytically active for the OCM reaction for at least 500 hours, preferably at least 1000 hours, or more preferably at least 1500 hours of use (time-on-stream or TOS). Notably, the conversion and selectivity parameters remain stable during such prolonged uses. These and other non-limiting aspects of the present invention are discussed in more detail in the following paragraphs.

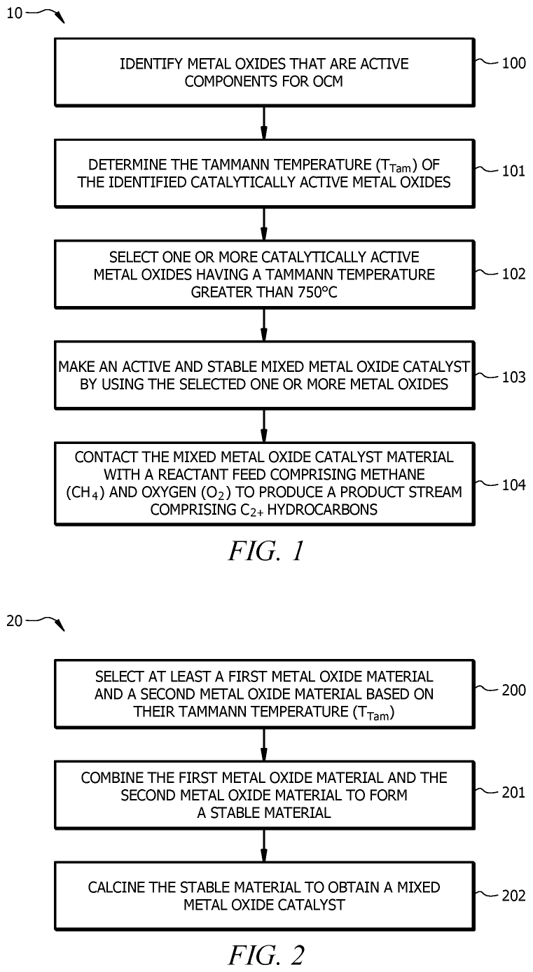

FIG. 1 shows method 10 for selecting a stable mixed metal oxide catalyst for an oxidative coupling of methane reaction, according to embodiments of the invention. Method 10 may start at block 100, which involves identifying metal oxide materials that are active components for the OCM reaction.

It has been discovered that selecting the appropriate mixed metal oxide material for use in the mixed metal oxide catalyst for the oxidative coupling of methane can result in a mixed metal oxide catalyst that is sufficiently stable to catalyze the oxidative coupling of methane reaction over periods not previously achieved. One aspect of the discovery is that one of the features that may be used in selecting the appropriate mixed metal oxide material is the Tammann temperature of at least one or more catalytically active metal oxide(s) in the mixed metal oxide material. The Tammann temperature is the temperature of a solid in degrees K that is sufficient to make atoms or ions of the bulk of the solid sufficiently mobile to cause bulk-to-surface migrations.

In method 10, block 101 may include determining (identifying) the Tammann temperature (T_{Tam}) of the catalytically active metal oxides identified in block 100. Identifying the Tammann temperature of the catalytically active metal oxides may involve testing the identified metal oxides or receiving previously determined Tammann temperature of the catalytically active metal oxides, for example, from publications.

Tables 1 and 2 show the Tammann and Wittig temperatures for various metal oxides that can be used for oxidative coupling of methane reaction. It should be noted that this list is exemplary only and embodiments of the invention are not limited to metal oxides selected from this list.

TABLE 1
Tammann and Huttig Temperature for Various
Metal Oxides used for Oxidative Coupling of Methane

| | Tammann | Hüttig |
|---|---|---|
| Metal Oxide | Temperature, C. | Temperature, C. |
| Magnesium oxide, MgO | 1290 | 665 |
| Lanthanum Oxide, La₂O₃ | 1021 | 503 |
| Cerium Oxide, CeO2 | 1064 | 529 |
| Samarium Oxide, Sm₂O₃ | 1031 | 509 |
| Thorium Oxide, ThO₂ | 1559 | 826 |
| Ytterbium Oxide, Yb₂O₃ | 1041 | 515 |
| Strontium Oxide, SrO | 1129 | 568 |
| Barium Oxide, BaO | 825 | 386 |
| Manganese Oxide, MnO | 836 | 392 |
| Manganese Oxide, Mn₃O₄ | 647 | 279 |
| Lead Oxide, PbO | 308 | 75 |

TABLE 2
Tammann and Hüttig temperature for various metal oxides

| Compound | T_{melting} | T_{Tammann} | T_{Hüttig} |
|---|---|---|---|
| Pt | 2028 | 1014 | 608 |
| PtO | 823 | 412 | 247 |
| PtO₂ | 723 | 362 | 217 |
| PtCl₂ | 854^{c} | 427 | 256 |
| PtCl₄ | 643^{c} | 322 | 193 |
| Pd | 1828 | 914 | 548 |
| PdO | 1023^{c} | 512 | 307 |
| Rh | 2258 | 1129 | 677 |
| Rh₂O₃ | 1373^{c} | 687 | 412 |
| Ru | 2723 | 1362 | 817 |
| Fe | 1808 | 904 | 542 |
| Co | 1753 | 877 | 526 |
| Ni | 1725 | 863 | 518 |
| NiO | 2228 | 1114 | 669 |
| NiCl₂ | 1281 | 641 | 384 |
| Ni(CO)₄ | 254 | 127 | 76 |
| NiS | 1249 | 625 | 375 |
| Ag | 1233 | 617 | 370 |
| Au | 1336 | 668 | 401 |
| Cu | 1356 | 678 | 407 |
| CuO | 1599 | 800 | 480 |
| Cu₂O | 1508 | 754 | 452 |
| CuCl₂ | 893 | 447 | 268 |
| Cu₂Cl₂ | 703 | 352 | 211 |
| Mo | 2883 | 1442 | 865 |
| MoO₃ | 1068 | 534 | 320 |
| MoS₂ | 1458 | 729 | 437 |
| Zn | 693 | 347 | 208 |
| ZnO | 2248 | 1124 | 675 |
| Al₂O₃ | 2318 | 1159 | 695 |
| SiO₂^{a} | 1986 | 993 | 596 |
| SiO₂^{b} | 1883 | 942 | 565 |

^{a} Crystobalite.
^{b} Quartz.
^{c} Decomposes at this temperature.
Source: J. A. Moulijn, A. E. van Diepen, F. Kapteijn “Catalyst Deactivation: is it Predictable? What to do?,” Applied Catalysis A: General 212(2001) 3-16.

In embodiments of the invention, by selecting metal oxide materials so that the one or more of the catalytically active metal oxides therein have a Tammann temperature higher than the operating temperature of the oxidative coupling of methane reaction, stable performance of the mixed metal oxide catalyst can be achieved. In embodiments of the invention, the operating temperature of the oxidative coupling of methane reaction may be about 750° C. Thus, at block 102, according to embodiments of the invention, method 10 may include selecting the one or more catalytically active metal oxide materials having a Tammann temperature greater than 750° C. In embodiments of the invention, the oxidative coupling of methane reaction operating temperature is 750° C. to 1100° C., most preferably 850° C. to 950° C. In embodiments of the invention, the Tammann temperature is no less than 10% or 20% of the oxidative coupling of methane reaction operating temperature. Therefore, in embodiments of the invention, for example, as may be dictated by the operating temperature of the oxidative coupling of methane reaction, the one or more catalytically active metal oxide materials that is selected for the mixed metal oxide catalyst may have a Tammann temperature that is greater than 850° C., preferably greater than 950° C., or more preferably greater than 1000° C., or 750° C. to 1700° C.

In embodiments of the invention, at least one or all of the catalytically active metal oxides in the metal oxide material can have a Tammann temperature of greater than 750° C. In embodiments of the invention, for example, as may be dictated by the operating temperature of the oxidative coupling of methane reaction, each of the metal oxides in the mixed metal oxide material may have a Tammann temperature greater than 750° C., preferably greater than 850° C., more preferably greater than 950° C., or even more preferably greater than 1000° C., or 750° C. to 1700° C.

It is possible that one or more components of a mixed metal oxide catalyst may have a Tammann temperature greater than a particular value, yet the Tammann temperature of the entire mixed metal oxide catalyst has a Tammann temperature below that value. In view of this, in embodiments of the invention, selections of the one or more of the metal oxides may be carried out such that the Tammann temperature of the mixed metal oxide material (as opposed to only the Tammann temperature of a given metal oxide that makes up the mixed metal oxide material) is above 750° C. In embodiments of the invention, the mixed metal oxide material has a Tammann temperature greater than 750° C., preferably greater than 850° C., more preferably greater than 950 ° C., or even more preferably greater than 1000° C., or 750° C. to 1700° C.

It is possible that one or more components of a mixed metal oxide catalyst material may have a Tammann temperature lower than a particular value, yet the Tammann temperature of the entire mixed metal oxide catalyst material has a Tammann temperature above that value. In view of this, in embodiments of the invention, selections of the one or more of the metal oxides may be carried out such that the Tammann temperature of the mixed metal oxide material (as opposed to only the Tammann temperature of a given metal oxide that makes up the mixed metal oxide material) is above 750° C. In embodiments of the invention, the mixed metal oxide material has a Tammann temperature greater than 750° C., preferably greater than 850° C., more preferably greater than 950° C., or even more preferably greater than 1000° C., or 750° C. to 1700° C.

Embodiments of the invention may use a SrCeYb oxide catalyst. Alternatively or additionally, embodiments of the invention may use other oxidative coupling of methane reaction catalysts such as mixed metal oxides selected from La₂O₃/CeO₂, SrO/La₂O₃, Li/MgO, etc. Further, embodiments of the invention may include any compounds (e.g., new crystalline phases) formed during catalyst synthesis and pretreatment (e.g., calcination).

In embodiments of the invention, for example, as may be dictated by the operating temperature of the oxidative coupling of methane reaction, the mixed metal oxide material can include metals from Column 1, Column 2, transitions metals, post-transition metals, or the lanthanides, and/or actinides of the Periodic Table. Non-limiting examples of Column 2 metals include magnesium (Mg), and/or strontium (Sr). Lanthanide metals can include lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and/or lutetium (Lu). Non-limiting examples of actinides include thorium. Transition metals can include Column 4 metals, for example, zirconium and titanium, and Column 12 metals, for example, zinc. Post-transition metals can include aluminum and silicon. In some instances, the mixed metal oxide material can be a mixture of La₂O₃ and another metal oxide from Column _{2,} lanthanides, or actinides of the Periodic Table. By way of example, ThO₂—La₂O₃, MgO—La₂O₃, SrO—La₂O₃, CeO₂—La₂O₃, Yb₂O₃—La₂O₃, Sm₂O₃—La₂O₃, or mixtures thereof can be used in the context of the present invention.

In embodiments of the invention, the mixed metal oxide material can include three catalytically active metal oxides selected from the actinides, alkaline earth metals (Column 2 metals), transition metals, lanthanides or mixtures thereof. By way of example, the three metals can be selected from thorium (Th), magnesium (Mg), strontium (Sr), cerium (Ce), ytterbium (Yb), samarium (Sm), lanthanum (La), erbium (Er), neodymium (Nd), dysprosium (Dy), gadolinium (Gd), europium (Eu), praseodymium (Pr), thulium (Tm), scandium (Sc), yttrbium (Yb), promethium (Pm), terbium (Tb), holmium (Ho), lutetium (Lu), zirconium (Zr), titanium (Ti), zinc (Zn), aluminum (Al), and silicon (Si). Non-limiting examples of mixed metal oxide materials having three active catalytic metals includes SrCeYb oxide, oxides of MgCeYb, MgCeLa, MgCePr, MgCeNd, MgCeSm, MgCeEu, MgCeGd, MgCeDy, MgCeEr, SrCeLa, SrCePr, SrCeNd, SrCeSm, SrCeEu, SrCeGd, SrCeDy, SrCeEr, BaCeYb, BaCeLa, BaCePr, BaCeNd, BaCeSm, BaCeEu, BaCeGd, BaCeDy, BaCeEr MgPrYb, MgPrLa, MgPrNd, MgPrSm, MgPrEu, MgPrGd, MgPrDy, MgPrEr, MgPrYb, SrPrYb, SrPrLa, SrPrNd, SrPrSm, SrPrEu, SrPrGd, SrPrDy, SrPrEr, SrPrYb, BaPrYb, BaPrLa, BaPrNd, BaPrSm, BaPrEu, BaPrGd, BaPrDy, BaPrEr, and BaPrYb. Other non-limiting examples of mixed metal oxides that may be used in embodiments of the invention include Sr_{0.9}Ce_{0.1}CoO₃₋ₓ, Sr_{0.9}Ce_{0.1}FeO₃₋ₓ, Sr_{0.9}Ce_{0.1}Co_{0.5}Fe_{0.5}O₃₋ₓ, Sr_{0.9}La_{0.1}Co_{0.5}Fe_{0.5}O₃₋ₓ, La_{0.9}Sr_{0.1}Ga_{0.8}Mg_{0.2}O₃₋ₓ, La_{0.9}Sr_{0.1}(Ga_{0.9}Fe_{0.1})_{0.8}Mg_{0.2}O₃₋ₓ, Sr_{1-y}Ce_{y}FeO₃₋ₓ, SrFeO_{2.80}, Sr_{0.9}Ce_{0.1}FeO_{2.78}, Sr_{0.8}Ce_{0.2}FeO_{2.795}, Sr_{0.7}Ce_{0.3}FeO_{2.82}, Ba_{1-y}Sr_{y}Sr_{y}Ce₁₋ₓYₓO_{3-δ}, BaCe_{0.9}Y_{0.7}O_{3-δ}, BaCe_{0.8}Y_{0.2}Y_{3-δ}, BaCe_{0.75}Y_{0.25}O_{3-δ}, BaCe_{0.7}Y_{0.3}O_{3-δ}, BaCe_{0.6}Y_{0.4}O_{3-δ}, BaCe_{0.5}Y_{0.5}O_{3-δ}, and (La,Sr)CeZrOx-based materials, where x≤0.5, y≤0.5, and δ≤0.5.

Referring to FIG. 1, method 10, at block 103, may involve making an active and stable mixed metal oxide catalyst by using the selected one or more metal oxides for the oxidative coupling of methane reaction. The mixed metal oxides may be made as described below in FIG. 2, method 20. Additionally or alternatively, block 103 may involve obtaining a mixed metal oxide material having catalytically active metal oxides for the oxidative coupling of methane reaction by acquiring the mixed metal oxide material from commercial sources such as those identified below.

Once the mixed metal oxide material has been made or obtained for use as the catalyst in the oxidative coupling of methane reaction as provided by block 103, method 10 may further include, at block 104, contacting the selected mixed metal oxide material with a reactant feed comprising methane (CH₄) and oxygen (O₂) to produce a product stream comprising C₂₊ hydrocarbons, wherein C₂₊ hydrocarbons can include a mixture of ethane, ethylene, and C₃ and higher hydrocarbons. In embodiments of the invention, the product stream may further comprise carbon dioxide (CO₂) and carbon monoxide (CO).

Based on the selection process described above, the OCM mixed metal oxide catalyst can be more stable than contemporary catalysts that are not so configured. In embodiments of the invention, contacting the selected mixed metal oxide material with the reactant feed is performed for greater than 500 hours without needing to regenerate the selected mixed metal oxide material. In embodiments of the invention, the oxygen (O₂) conversion is greater than 70% or greater than 90% after 500 hours, preferably 1000 hours, or more preferably greater than 1500 hours, time on the stream. In embodiments of the invention, the C₂₊ hydrocarbon selectivity is greater than 60% or 60% to 85% after 500 hours, preferably 1000 hours, or more preferably greater than 1500 hours, time on the stream.

As noted above, the individual metal oxides and/or the mixed metal oxide catalyst described in method 10 may be acquired from sources such as Sigma-Aldrich® (U.S.A.) or Fisher Scientific, or Alfa Aesar, or any other commercial sources. Additionally or alternatively, the metal oxides can be made using precipitation, co-precipitation, or sol-gel methodology. FIG. 2 shows method 20, which may be used to make the mixed metal oxide catalyst for oxidative coupling of methane, according to embodiments of the invention. Method 20 may begin at block 200, which involves selecting at least a first metal oxide material and a second metal oxide material based on the Tammann temperature (T_{Tam}) of the corresponding metal oxide. In method 20, the T_{Tam} of at least one metal oxide is greater than 750° C. After selecting the first and second metal oxide material, at block 201, the first metal oxide material and the second metal oxide material are combined to form a stable material. Once formed, the stable material is calcined to obtain a mixed metal oxide catalyst, at block 202. Calcining may include subjecting the material to a temperature greater than 350° C., preferably greater than 800° C. in the presence of an oxygen source, preferably air.

The first and second metal oxide materials may be metal salts or metal oxide precursors, and in that scenario combining the first metal oxide material and the second metal oxide material at block 201 may involve obtaining a solution comprising first and second metal salts and drying the solution at 110° C. to 130° C. to obtain a stable mixture. The stable mixture can be heated in the presence of an oxidant (e.g., calcined in air) to convert the stable mixture to a mixed metal oxide. By way of example, the stable mixture can be heated at 350° C. to 800° C. under a flow of air. Alternatively or additionally, when the first and second metal oxide materials are metal salts, combining the first metal oxide material and the second metal oxide material at block 201 may involve obtaining a first solution comprising the first metal salt and a second solution comprising the second metal salt. The first solution may then be added to the second solution to precipitate a mixed metal salt. Subsequently, the method may involve drying the solution at 110° C. to 130° C. to obtain the stable mixture. The stable mixture can be calcined at 350 to 800° C. to convert the mixed metal salt to mixed metal oxide catalyst. Alternatively or additionally, when the first and second metal oxide materials are metal oxides, combining the first metal oxide material and the second metal oxide material at block 201 may involve mixing and pulverizing the metal oxides to form the stable material.

### EXAMPLE

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results. Experiments were performed to show the performance of the oxidative coupling of methane reaction in temperature-programmed sequences. The experiments show nearly stable performance of SrCeYb Oxide catalyst at the following feed conditions: feed CH₄/O₂ ratio of 7.4, residence time of 1.4 ms. Table 3 shows the results of the experiment at these conditions. The mass of converted methane per gram of catalyst was in excess of 33,750 g.

TABLE 3
Performance of the SrCeYb Oxide catalyst at various times on stream
(Conditions: Feed CH₄/O₂ ratio of 7.4, residence time of 1.4 ms)

| | Time on stream, hours | | | |
|---|---|---|---|---|
| | 167 | 181 | 942 | 1642 |
| | Temperature, ° C. | | | |
| | 750 | 775 | 800 | 813 |
| CH₄ Conversion | 14.5 | 16.6 | 16.2 | 15.1 |
| O₂ Conversion | 80.0 | 92.4 | 88.7 | 84.5 |
| ‘C’ Selectivities | | | | |
| C₂═ (ethylene) | 23.1 | 28.5 | 28.0 | 29.7 |
| C₂≡ (acetylene) | 0.1 | 0.2 | 0.2 | 0.4 |
| C₂ (ethane) | 45.3 | 43.2 | 41.9 | 39.4 |
| C₃═ (propylene) | 1.3 | 1.5 | 1.6 | 2.0 |
| C₃ (propane) | 2.8 | 2.2 | 1.8 | 1.7 |
| C₄═ (butylene, 1-butene, 2-butene) | 0.7 | 1.0 | 0.8 | 0.9 |
| % C₂+ | 73.3 | 76.7 | 74.4 | 74.0 |
| % CO | 2.9 | 1.7 | 2.6 | 2.5 |
| % CO₂ | 23.8 | 21.6 | 23.0 | 23.5 |

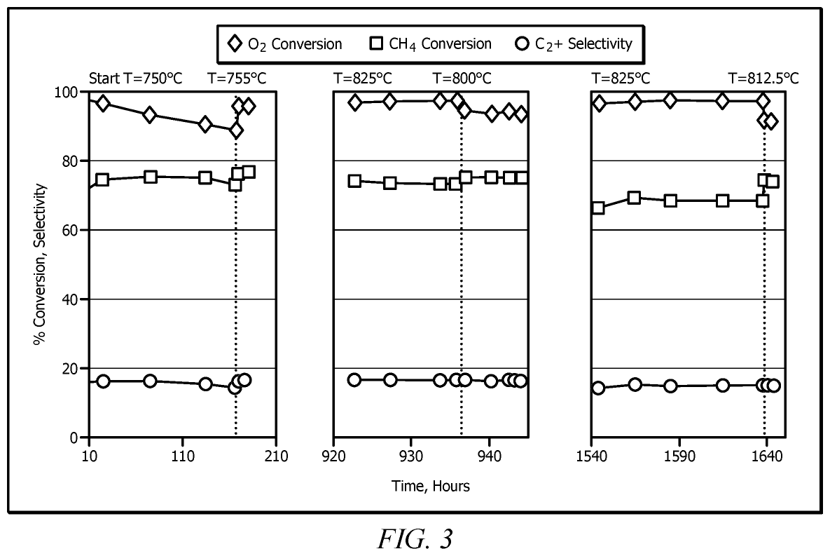

FIG. 3 shows the performance of the reaction in temperature-programmed sequences. FIG. 3 illustrates the performance of SrCeYb oxide catalyst with time on stream (Feed CH₄/O₂ ratio=7.4; Residence Time=1.4 ms).

1. A method of selecting a stable mixed metal oxide catalyst for an oxidative coupling of methane (OCM) reaction, the method comprising:
(a) obtaining a mixed metal oxide material having catalytically active metal oxides for the OCM reaction;
(b) identifying the Tammann temperature (T_{Tam}) of at least one of the catalytically active metals oxides of the mixed metal oxide material; and
(c) selecting the mixed metal oxide material for use as a catalyst in the OCM reaction if the at least one catalytically active metal oxides present in the mixed metal oxide material has a T_{Tam} greater than 750° C.

2. The method of claim 1, wherein the T_{Tam} for the at least one catalytically active metal oxide is greater than 850° C., preferably greater than 950° C., or more preferably greater than 1000° C., or 750° C. to 1700° C.

3. The method of claim 1, wherein each of the metal oxides in the mixed metal oxide material has a T_{Tam} greater than 750° C.

4. The method of claim 1, wherein the T_{Tam} of the mixed metal oxide material is above 750° C.

5. The method of claim 1, wherein the mixed metal oxide material has two catalytically active metal oxides having a metal selected from the group consisting of thorium (Th), magnesium (Mg), strontium (Sr), cerium (Ce), ytterbium (Yb), samarium (Sm), and lanthanum oxide (La₂O₃).

6. The method of claim 1, wherein the mixed metal oxide material has three catalytically active metal oxides having a metal selected from the group consisting of thorium (Th), magnesium (Mg), strontium (Sr), cerium (Ce), ytterbium (Yb), samarium (Sm), lanthanum (La), erbium (Er), neodymium (Nd), dysprosium (Dy), gadolinium (Gd), europium (Eu), praseodymium (Pr), thulium (Tm), scandium (Sc), yttrbium (Yb), promethium (Pm), terbium (Tb) holmium (Ho), lutetium (Lu), zirconium (Zr), titanium (Ti), zinc (Zn), aluminum (Al), silicon (Si).

7. The method of claim 1, wherein the OCM reaction operating temperature is 750° C. to 1100° C., most preferably 850° C. to 950° C.

8. The method of claim 7, wherein the T_{Tam} is no less than 10% or 20% of the OCM reaction operating temperature.

9. The method of claim 1, wherein each catalytically active metal oxide present in the mixed metal oxide material is chemically inert with respect to components present in a product stream produced from the OCM reaction.

10. The method of claim 9, wherein the components in the product stream include C2+ hydrocarbons, carbon dioxide (CO2), and carbon monoxide (CO).

11. The method of claim 1, further comprising:
(d) contacting the selected mixed metal oxide material with a reactant feed comprising methane (CH₄) and oxygen (O₂) to produce a product stream comprising C₂+ hydrocarbons, wherein C₂+ hydrocarbons comprises mixture of ethane, ethylene and C₃ and higher hydrocarbons.

12. The method of claim 11, wherein reaction step (d) is performed for greater than 500 hours without regenerating the selected mixed metal oxide material.

13. The method of claim 11, wherein the oxygen (O₂) conversion is greater than 70% or greater than 90% after 500 hours, preferably greater than 1500 hours, time on the stream.

14. The method of claim 11, wherein the C₂+ hydrocarbon selectivity is greater than 60% or 60% to 85% after 500 hours, preferably greater than 1500 hours, time on the stream.

15. The method of claim 11, wherein the product stream further comprises carbon dioxide (CO₂) and carbon monoxide (CO).

16. A method of making a stable mixed metal oxide catalyst for an oxidative coupling of methane (OCM) reaction, the method comprising:
(a) selecting at least a first metal oxide material and a second metal oxide material based on the Tammann temperature (T_{Tam}) of the corresponding metal oxides thereof, wherein the T_{Tam} of at least one corresponding metal oxide is greater than 750° C.; and
(b) combining the first metal oxide material and the second metal oxide material to form a stable material;
(c) calcining the stable material to obtain a mixed metal oxide catalyst.

17. The method of claim 16, wherein the first and second metal oxide materials are metal salts, and wherein combining in step (b) comprises:
obtaining a solution comprising the first and second metal oxide metal salts; and
(ii) drying the solution at 110° C. to 130° C. to obtain a stable mixture.

18. The method of claim 16, wherein the first and second metal oxide materials are metal salts, and wherein combining in step (b) comprises:
(i) obtaining a first solution comprising the first and a second solution comprising the second metal oxide metal salts; and
(ii) adding the first solution to the second solution to precipitate the mixed metal oxide;
(iii) drying the solution at 110° C. to 130° C. to obtain the stable mixture.

19. The method of claim 16, wherein the first and second metal oxide materials are metal oxides, and wherein combining in step (b) comprises pulverizing the metal oxides to form the stable material.

20. The method of claim 15, wherein step (d) calcining comprises subjecting the material to a temperature greater than 350° C., preferably great than 800° C. in the presence of an oxygen source, preferably air.
